(19) United States
(12) Patent Application Publication
(10) Pub. No.: US 2026/0325822 A1
(43) Pub. Date: Oct. 22, 2026

(54) ROTOR AND FLUID PUMP FOR CARDIAC ASSISTANCE

(71) Applicant: Berlin Heart GmbH, Berlin (DE)

(72) Inventors: Evgenii Palamarchuk, Berlin (DE); Bodo Schmidt, Berlin (DE)

(73) Assignee: Berlin Heart GmbH, Berlin (DE)

(21) Appl. No.: 19/099,536

(22) PCT Filed: Jul. 27, 2023
(86) PCT No.: PCT/EP2023/070936

§ 371 Date: Jan. 29, 2025

(30) Foreign Application Priority Data
Jul. 29, 2022 (EP) 22187820.0

(51) Int. Cl. A61M 60/232; A61M 60/178; A61M 60/422; A61M 60/806; A61M 60/82

(52) U.S. Cl. CPC A61M 60/232; A61M 60/178; A61M 60/422; A61M 60/806; A61M 60/82

(57) ABSTRACT

A fluid pump for cardiac support and a rotor for a fluid pump are provided. A rotor may comprise a rotor magnet arrangement for contactlessly supporting the rotor within a cavity of a fluid pump, a hub structure comprising one or more magnets and arranged at a first end of the rotor and centered about the axis of rotation, and a base plate having a blading for conveying fluid, where intermediate spaces of the blading form fluid channels which open into respective outlet openings and are delimited towards a second end of the rotor by the base plate. At least one of the fluid channels may have a passage opening connecting the fluid channel to the cavity, the passage opening extending between the outlet opening and the hub structure along at least part of a first side of the fluid channel, the first side facing the first end.

### CROSS-REFERENCE TO RELATED APPLICATIONS

This application is a U.S. national stage application under 35 U.S.C. § 371 of PCT Application No. PCT/EP2023/070936, filed on Jul. 27, 2023, and published on Feb. 1, 2024 as WO 2024/023272A1 , which claims priority to European Patent Office Application No. EP 22187820.0, filed on Jul. 29, 2022. The entire contents of each of the above applications is incorporated herein by reference.

### BRIEF DESCRIPTION OF THE DRAWINGS

Exemplary embodiments of the subject matter of the application are explained below with reference to drawings. The drawings show schematically:

FIG. 1A: a longitudinal section of a fluid pump according to an example,

FIG. 1B and FIG. 1C: perspective views of a rotor of the fluid pump shown in FIG. 1A,

FIG. 2: a perspective view of a rotor not corresponding to the subject matter of the application,

FIG. 3A and FIG. 3B: characteristic curves of fluid pumps with rotors of the types shown in FIGS. 1A-1C and FIG. 2,

FIG. 4A and FIG. 4B: perspective views of a rotor according to a further example,

FIG. 5A and FIG. 5B: perspective views of a rotor according to a further example,

FIG. 6A: a perspective view of components of a rotor according to a further example,

FIG. 6B: a further perspective view of one of the components shown in FIG. 6A,

FIG. 7A: a perspective view of components of a rotor according to a further example,

FIG. 7B: a further perspective view of one of the components shown in FIG. 7A,

FIG. 8A: a perspective view of components of a rotor according to a further example,

FIGS. 8B and 8C: further views of the components shown in FIG. 8A.

Recurring and similar features of different embodiments are marked with identical alphanumeric reference symbols in the drawings. Reference signs already explained or shown in connection with another drawing may be omitted.

### DETAILED DESCRIPTION

The application relates to a rotor for a fluid pump for cardiac support and a corresponding fluid pump. The subject matter of the application is particularly applicable in the field of short-term or long-term cardiac support by means of implantable fluid pumps, in particular VADs (VAD stands for ventricular assist device).

Various rotary fluid pumps for cardiac support are known in the state of the art, in particular those that may be used as implantable VADs fluidically connectable to an existing heart.

Rotors of such fluid pumps often have complex geometries that require correspondingly complex manufacturing processes. For example, rotor geometries with cavities inside the rotor may require multi-piece manufacturing, i.e. manufacturing from several individual parts, and assembly of the individual parts and/or subsequent surface treatment to ensure sufficient surface quality. For example, a rotor formed in several pieces by welding may require reworking of weld seams, which may be made more difficult by the limited accessibility of any cavities. These aspects may also make it difficult to comply with production tolerances.

For rotors of fluid pumps, it is also desirable to satisfy certain parameter specifications, for example with regard to certain characteristic curves of the rotor and/or the fluid pump. For example, a dp-Q characteristic curve may be desirable that remains within a predetermined range and/or has a slope, at least in part, that is greater than or equal to a predetermined minimum slope. The dp-Q characteristic curve describes a pressure difference (dp) across the fluid pump or a part of the fluid pump, said part in particular comprising the rotor, as a function of a volume flow (Q) through the fluid pump. A suitable dp-Q characteristic curve may prevent or reduce the occurrence of volume flows that are too low or negative (i.e. opposite to the pumping direction). A suitable dp-Q characteristic curve may also enable, for example, a reliable estimation of the volume flow using a measured pressure difference or a pressure difference determined from measured data, which may be advantageous for controlling the fluid pump (open-loop or closed-loop control), for example using a zero-force controller of the type described below. Furthermore, an axial force characteristic curve that remains within a predetermined range and/or is at least partially monotonic, preferably strictly monotonic and/or as steep as possible, may be desirable. The axial force characteristic curve describes an axial force acting along an axis of rotation as a function of a volume flow through the fluid pump. A suitable axial force characteristic curve may, for example, enable a reliable estimation of the volume flow using a measured axial force and/or axial position or one determined from measured data, which in turn may be advantageous for controlling the fluid pump (open-loop or closed-loop control), for example using a zero-force controller.

Against the background of the prior art, the present application is based on the problem of providing a rotor for a fluid pump for cardiac support and a corresponding fluid pump, both having at least some of the desired properties mentioned.

The problem is solved by proposing a rotor for a fluid pump for cardiac support according to the application, and a fluid pump for cardiac support according to the application. Further developments and preferred embodiments are provided based on the features described in the application.

The proposed rotor for a fluid pump for cardiac support comprises a rotor magnet arrangement for contactlessly supporting the rotor within a cavity of the fluid pump such that the rotor is rotatable about an axis of rotation for conveying a fluid from an inlet of the cavity to an outlet of the cavity, and a first end of the rotor is disposed closer to the inlet than a second end of the rotor opposite the first end along the axis of rotation, a hub structure arranged at the first end and centered about the axis of rotation, the hub structure comprising one or more magnets of the rotor magnet arrangement, a base plate arranged at the second end or between the hub structure and the second end and having a blading for conveying the fluid, wherein intermediate spaces of the blading form fluid channels which open into respective outlet openings and are delimited towards the second end by the base plate.

The fluid pump (also referred to as a pump for short) is a rotary fluid pump, which may in particular be a centrifugal pump, alternatively an axial pump or a mixed form (e.g. a pump in which the pumped fluid exits the outlet of the cavity with both axial and radial flow components during operation). The rotor and the pump are configured in particular for conveying blood as a fluid, for which purpose the inlet and the outlet of the cavity may be fluidically connected or connectable to a heart and/or to a blood vessel connected to the heart.

The rotor may preferably be contactlessly magnetically supportable in the cavity from all sides; in addition to magnetic forces, other types of forces, such as hydrodynamic forces, may contribute to supporting the rotor in the cavity.

The rotor magnet arrangement is configured to interact magnetically with a stator magnetic bearing arrangement of the fluid pump to support the rotor in the cavity. In particular, the rotor magnet arrangement and the stator magnetic bearing arrangement may form an active magnetic bearing along at least a first direction, i.e. a magnetic bearing in which a magnetic field may be varied along the first direction by controlling electromagnets in order to exert a variable bearing force on the rotor. Alternatively, the rotor magnet arrangement and the stator magnetic bearing arrangement may form a passive magnetic bearing along the first direction. The rotor magnet arrangement and the stator magnetic bearing arrangement may be configured to passively magnetically or actively magnetically support the rotor along further spatial directions.

The rotor is configured to form an electric motor together with a stator of the fluid pump, so that the rotor may be set in rotation around the axis of rotation to convey the fluid. In particular, permanent magnets of the rotor magnet arrangement may form a motor, such as a brushless DC motor, together with coils of the stator magnetic bearing arrangement and/or coils of a stator motor arrangement. In particular, the stator and the rotor may form an axial flux motor; alternatively, the stator and the rotor may form a radial flux motor, for example.

Due to the hub structure arranged at the first end, a particularly stable magnetic bearing of the rotor in the cavity may be achieved, whereby the magnet or magnets of the rotor magnet arrangement comprised by the hub structure may have a stabilizing effect against tilting of the rotor with respect to the axis of rotation, among other things.

The actual axis of rotation may be shifted and/or tilted, particularly during operation, in accordance with a movement of the rotor. However, when describing arrangements and orientations of elements with regard to the axis of rotation, it is assumed in the following that the axis of rotation is fixed and corresponds to a fixed longitudinal axis of the pump housing.

The hub structure may protrude from the base plate in the direction of the first end. A length of the hub structure projecting from the base plate along the axis of rotation may, for example, be at least a quarter, at least a third or at least half of a total length of the rotor along the axis of rotation; in particular, the length of the hub structure projecting from the base plate along the axis of rotation may be between 40 percent and 45 percent or between 35 percent and 40 percent of the total length of the rotor along the axis of rotation. An outer width of the hub structure perpendicular to the axis of rotation may, for example, be at least one third or at least half, in particular approx. 50 percent, of a total width of the rotor perpendicular to the axis of rotation. The hub structure is preferably essentially rotationally symmetrical with respect to the axis of rotation. In particular, the hub structure may be essentially cylindrical in at least some sections and/or essentially conical in at least some sections; the hub structure may have a variable cross-section along the axis of rotation.

The hub structure may comprise one or more cavities for accommodating the magnet or magnets of the rotor magnet arrangement comprised by the hub structure. One or more of the one or more magnets of the rotor magnet arrangement comprised by the hub structure may be configured to cooperate with a control coil of the stator magnetic bearing arrangement for exerting a force, in particular a force along the axis of rotation, on the rotor. One or more of the one or more magnets of the rotor magnet arrangement encompassed by the hub structure may be arranged to cooperate with a motor coil of the stator magnetic bearing arrangement and/or a stator motor assembly for exerting a torque with respect to the axis of rotation on the rotor and/or for exerting a force, in particular a force along the axis of rotation, on the rotor.

The base plate may be essentially disc-shaped; for example, the base plate may essentially have the shape of a flattened cylinder. The base plate may form or comprise a region extending fully around the axis of rotation. In particular, the base plate or the region extending fully around the axis of rotation may essentially have the shape of a cylinder or a cylinder ring.

The base plate may comprise one or more additional magnets of the rotor magnet arrangement. The base plate may comprise one or more cavities for accommodating the magnet or magnets of the rotor magnet arrangement comprised by the base plate. One or more of the one or more magnets of the rotor magnet arrangement comprised by the base plate may be configured to cooperate with the stator magnet arrangement, in particular with a control coil of the stator magnetic bearing arrangement, for exerting a force, in particular a force along the axis of rotation, on the rotor, in particular for contactlessly supporting the rotor within the cavity. One or more of the one or more magnets of the rotor magnet arrangement comprised by the base plate may be arranged to cooperate with a motor coil of the stator magnetic bearing arrangement for exerting a torque with respect to the axis of rotation on the rotor and/or for exerting a force, in particular a force along the axis of rotation, on the rotor.

The blading preferably comprises a plurality of blades, with the fluid channels being formed between respective side surfaces of adjacent blades facing each other. The blades may extend essentially radially from a central area of the base plate to an outer area of the base plate, whereby the blades are preferably angled and/or spirally curved in relation to the radial direction. The blading may be configured to provide the conveyed fluid with a radial flow component (in the direction of the outlet opening) during operation. The blades may be angled and/or curved in relation to the axis of rotation.

It is provided that at least one of the fluid channels has a passage opening connecting the fluid channel to the cavity, the passage opening extending between the outlet opening and the hub structure along at least part of a first side of the fluid channel facing the first end.

The resulting geometry of the fluid channels, which is at least partially open towards the first end (in the area of the passage openings) and closed towards the second end (delimited by the base plate), is also referred to here as a semi-open rotor geometry and the corresponding rotor as a semi-open rotor. A geometry of the fluid channels that is also closed towards the first end (e.g. delimited by a cover plate opposite the base plate) is also referred to here as a closed rotor geometry and the corresponding rotor as a closed rotor.

By providing said passage openings, manufacturing of the rotor may be simplified and/or compliance with specified manufacturing tolerances may be facilitated. In particular, this makes it possible to integrally (in one piece) manufacture a component of the rotor, which would have to be manufactured in several pieces if the respective passage opening were missing. In particular, splits or seams in the blading area may be avoided. Due to the rotor geometry, which is open towards the first end in the area of the passage opening, the base plate or at least part of the base plate may be milled or cast integrally together with the blading or part of the blading. This rotor geometry, which is open towards the first end, may also provide access for reworking, such as polishing or grinding, of surfaces of the rotor, particularly in the area of the fluid channels.

The provision of the passage openings also provides a parameter space for the design of the rotor or the fluid pump, within which certain parameter specifications may be achieved and/or operating parameters of the fluid pump may be adapted and/or optimized compared to other rotor geometries. For example, it has been recognized that a steepness of a dp-Q characteristic curve may be increased over certain ranges in a rotor with passage openings, i.e. with a rotor geometry open towards the first end, compared to a rotor with an otherwise comparable design, but without passage openings, i.e. with a rotor geometry closed towards the first end (cf. for example FIG. 3A and its description further below). It has also been recognized that an axial force characteristic curve for a rotor with a rotor geometry that is open towards the first end may be steeper in some areas and monotonic over a larger area compared to a rotor with an otherwise comparable design but with a rotor geometry that is closed towards the first end (see, for example, FIG. 3B and its description further below).

Preferably, several fluid channels arranged at regular intervals around the axis of rotation, in particular all fluid channels, each have a passage opening of the type described.

It may be provided that the passage opening of at least one, preferably each, of the fluid channels extends along at least a part, in particular along only a part, of an extension of the fluid channel from the outlet opening in the direction of the hub structure. It may be provided that the passage opening of at least one, preferably each, of the fluid channels extends along the entire extension of the fluid channel from the outlet opening to the hub structure. By adjusting the extent to which the passage openings extend along the extension of the fluid channels, operating parameters of the pump and/or the rotor may be adjusted within the above-mentioned parameter space and/or balanced against each other and with regard to the achievable simplification of rotor production. If the passage opening only extends along part of the aforementioned extension of the fluid channel, possible blood damage during operation and/or susceptibility of the rotor position to externally acting torques and/or movements of the pump caused by external influences (such as rotations of the patient) may be limited, for example.

A corresponding effect may also be achieved by partially limiting the fluid channels towards the first end by means of projections of the blading (also known as winglets). Thus, at least one, preferably each, of the fluid channels on the first side at the level of the passage opening may be partially delimited by a projection of a surface of the blading adjacent to the respective fluid channel. The projection may project from the surface of the blading adjacent to the respective fluid channel against a direction of rotation of the rotor and/or said surface may have a concave curvature (in a plane perpendicular to the axis of rotation). The aforementioned effects of the projection may be particularly pronounced in this case. The projection may project in a direction of rotation of the rotor from the surface of the blading adjacent to the respective fluid channel and/or said surface may have a convex curvature (in a plane perpendicular to the axis of rotation). In this case, for example, manufacturing the rotor may be relatively simple due to the good accessibility of the convex curved surface.

The projection may be formed, for example, as a flat cover piece projecting perpendicularly from the adjacent surface of the blading. Alternatively, the projection may be formed as a continuous extension of the shape of a respective blade of the blading. The blade and/or the projection may be at least partially curved in a tangential plane parallel to the axis of rotation. This curvature results in a projecting blade tip, which acts as a projection with the advantages described. The blades or projections may be curved in the tangential plane in such a way that they are convexly bulging in or against the direction of rotation of the rotor. The projection formed as a continuous extension of the blade shape may reduce inertia of the rotor with respect to rotation about the axis of rotation and further increase the efficiency of the rotor compared to the design with a perpendicularly projecting cover piece by suppressing the overflow vortex at the blade tip; blood damage may also be reduced by the same mechanisms as described above. A projecting blade tip on a pressure side of the blade (convex surface) is advantageous for production, as this side is more accessible for the tool. Depending on geometry parameters, a projecting blade tip on the suction side (concave surface) may result in a greater increase in efficiency. In addition, such a curvature (compared to the “cover piece” design) reduces or eliminates a pronounced edge, which could otherwise lead to comparatively higher local shear rates and resulting blood damage (and/or activation).

The hub structure may comprise an inlet channel extending along the axis of rotation and open at the first end, the inlet channel fluidically connected within the rotor to the fluid channels. The hub structure may thus act as a fluid inlet of the rotor, through which the fluid entering through the inlet of the cavity is directed to the blading. In particular, the fluid channels may have inlet openings opening into the inlet channel.

A transition from the blading to the hub structure may be rounded. A rounded transition may be preferable from a manufacturing point of view and/or result in improved hydrodynamic behavior, such as reduced turbulence and/or reduced blood damage.

A component of the rotor comprising the blading may be integrally formed, in particular by milling from a continuous piece of material. The integrally formed component comprising the blading may include at least a part of the base plate and/or at least a part of the hub structure. The hub structure may be formed as a separate hub assembly connectable or connected to the component comprising the blading. The hub structure and the component comprising the blading may comprise complementary alignment elements, which may form a flange connection and/or a groove connection and/or a pin connection, for example.

The rotor may be composed or composable of a first component, comprising at least a part of the hub structure, and at least one second component. The rotor may be composed or composable of said first component, a second component comprising at least one further part of the hub structure, and at least one third component comprising at least part of the base plate.

The base plate may comprise a central recess for contactlessly receiving a part of the stator, in particular a trunnion of the stator. Said part or trunnion may comprise a magnetic bearing component, in particular one or more magnets of the stator magnetic bearing arrangement, of the fluid pump. Magnets of the rotor magnet arrangement that are comprised by the base plate may be arranged peripherally to the central recess.

The proposed fluid pump for cardiac support comprises a rotor of the type proposed above and a stator with a cavity for receiving the rotor and a stator magnetic bearing arrangement which, together with the rotor magnet arrangement, forms a magnetic bearing, in particular an active magnetic bearing, for contactlessly supporting the rotor within the cavity.

The proposed rotor provides its above-mentioned effects and advantages in the proposed fluid pump. The fluid pump may be further implemented according to the optional features of the rotor described above. The rotor may include features that correspond to optional features of the fluid pump.

The pump may comprise a control unit for controlling (open-loop and/or closed-loop control) a rotation and/or position of the rotor within the cavity and/or may be connectable or connected to such a control unit. The pump may have one or more sensors for providing measurement signals corresponding to parameters of the rotation and/or position of the rotor and/or corresponding to other parameters, such as a pressure and/or a pressure difference and/or forces and/or torques acting on the rotor. The control unit may be configured to control (open-loop and/or closed-loop) the rotation and/or position of the rotor based on the measurement signals.

For example, the control unit may be configured to detect a measurement signal that may be registered at the motor coils, the measurement signal having a component that is dependent on the position and/or rotation of the rotor; the component may correspond in particular to a back electromotive force (Back-EMF) induced by the rotation of the rotor. In this way, it is possible to provide a measurement signal that may be used in particular for controlling (open-loop and/or closed-loop control) the rotation and/or position of the rotor without additional sensors.

Furthermore, the control unit may, for example, be configured to adjust the rotor to a target bearing position (in particular a variable position) at which external forces acting on the rotor along a bearing direction of action, for example along the axis of rotation, add up to a predetermined force, in particular a zero force, and/or at which the power used to actuate the control and/or motor coils is minimal. This type of control, also known as zero-force control, enables energy-efficient operation of the pump.

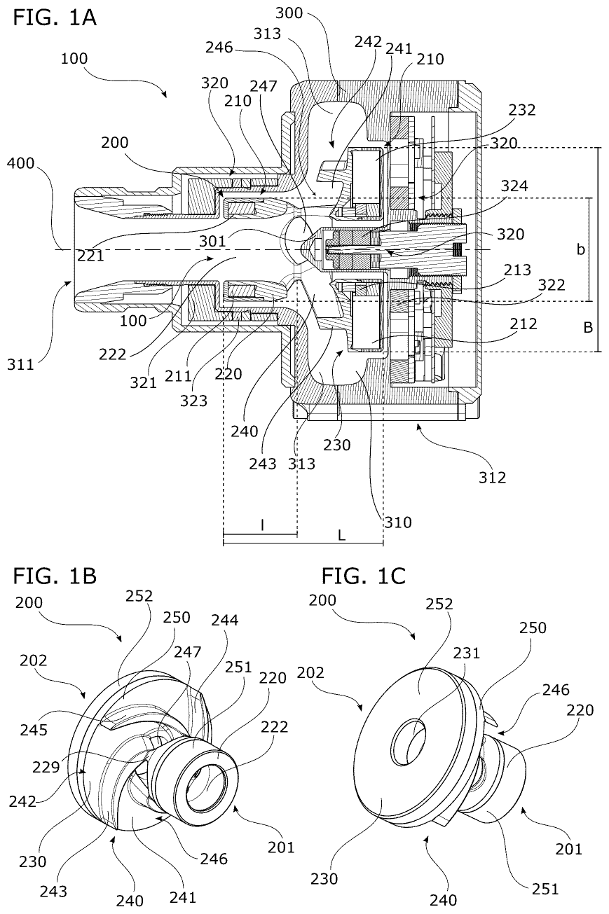

The fluid pump 100 shown in FIG. 1A comprises a rotor 200 (shown in further views in FIG. 1B and FIG. 1C) and a stator 300 formed as a housing. The stator 300 comprises a cavity 310 for accommodating the rotor 200.

The rotor 200 comprises a rotor magnet arrangement 210 for contactlessly supporting the rotor 200 within the cavity 310 such that the rotor 200 is rotatable about an axis of rotation 400 for conveying a fluid from an inlet 311 of the cavity to an outlet 312 of the cavity, and a first end 201 of the rotor 200 is disposed closer to the inlet 311 than a second end 202 of the rotor 200 opposite the first end 201 along the axis of rotation 400. Features of the fluid pump 100 arranged closer to the first end 201 or the inlet 311 than to the second end 202 or the outlet 312 are also referred to as inlet-side features, and features of the fluid pump 100 arranged closer to the second end 202 or the outlet 312 than to the first end 201 or the inlet 311 are also referred to as outlet-side features.

The rotor 200 further comprises a hub structure 220 arranged at the first end 201 and centered about the rotation axis 400, which comprises one or more inlet-side rotor bearing magnets 211 of the rotor magnet arrangement 210. For example, one or more ring-shaped and/or segmented inlet-side rotor bearing magnets 211 may be provided.

The rotor 200 further comprises a base plate 230 arranged at the second end 202 with a blading 240 for conveying the fluid, wherein intermediate spaces of the blading form fluid channels 241 which open into respective outlet openings 242 and are delimited towards the second end 202 by the base plate 230. The base plate 230 may alternatively be disposed between the hub structure 220 and the second end 202. The base plate 230 comprises a central recess 231 for contactlessly receiving a trunnion 301 of the stator 300.

The fluid pump 100 is a rotary fluid pump designed as a centrifugal pump. Alternatively, the fluid pump may also be an axial pump or a hybrid form. The rotor 200 and the fluid pump 100 are arranged to convey blood as the fluid, for which purpose the inlet 311 and the outlet 312 of the cavity 310 are fluidically connectable or connected to a heart and/or to a blood vessel connected to the heart. The cavity 310 comprises a volute 313, in which fluid emerging from the fluid channels 241 is collected and fed to the outlet 312.

The rotor 200 may be contactlessly magnetically supported in the cavity 300 on all sides; in addition to magnetic forces, other types of forces, such as hydrodynamic forces, may contribute to supporting the rotor 200 in the cavity 300.

The stator 300 comprises a stator magnetic bearing arrangement 320 which, together with the rotor magnet arrangement 210, forms a magnetic bearing for contactlessly supporting the rotor within the cavity. The rotor magnet arrangement 210 is correspondingly configured to interact magnetically with the stator magnetic bearing arrangement 320 for supporting the rotor 200 in the cavity. In particular, the rotor magnet arrangement 210 and the stator magnetic bearing arrangement 320 form an active magnetic bearing along the axis of rotation 400. The rotor magnet arrangement 210 and the stator magnetic bearing arrangement 320 are further arranged to passively magnetically support the rotor 200 in directions perpendicular to the axis of rotation 400; alternatively, passive magnetic support along the axis of rotation and/or active magnetic support in directions perpendicular thereto may also be provided. In the example described, the rotor magnet arrangement 210 comprises permanent magnets, and the stator magnetic bearing arrangement 320 comprises both electromagnets with coils and permanent magnets. Other combinations of permanent magnets and/or inducible magnets and/or electromagnets may also be provided.

The rotor 200 is configured to form a brushless DC motor together with the stator 300 of the fluid pump 100, so that the rotor 200 may be set in rotation about the axis of rotation 400 to convey the fluid. For this purpose, the rotor comprises The stator 200 and the rotor 300 form an axial flux motor; alternatively, the stator and the rotor may form another type of motor, such as a radial flux motor.

The inlet-side rotor bearing magnet or magnets 211 are arranged to interact with at least one control coil 321 of the stator magnetic bearing arrangement 320 arranged on the inlet side to exert a force along the axis of rotation 400 on the rotor 200 (part of the active axial magnetic bearing), whereby the force may have an attracting or repelling effect depending on the energization of the control coil 321. The rotor magnet arrangement 210 further comprises one or more outlet-side rotor motor magnets 212 configured to cooperate with outlet-side motor coils 322 of the stator magnetic bearing arrangement 320 for exerting a torque with respect to the axis of rotation 300 on the rotor 200 (part of the axial flux motor). Alternatively or additionally, a separate stator motor arrangement with motor coils may be provided. In addition to interacting with the motor coils 322 and/or other parts (shown here or additional) of the stator magnetic bearing arrangement 320, the rotor motor magnets 212 may be arranged to exert a force along the axis of rotation 400 (part of the active axial magnetic bearing), wherein the force may be attractive or repulsive depending on the energization of the motor coils 322. The rotor motor magnets 212 are arranged in the base plate. The base plate 230 may alternatively or additionally comprise one or more further magnets of the rotor magnet arrangement 210, which may in particular be arranged to cooperate with the motor coils 322 and/or other parts (shown here or additional) of the stator magnetic bearing arrangement 320 for exerting a force along the axis of rotation 400.

The inlet-side rotor bearing magnet or magnets 211 together with one or more inlet-side stator bearing magnets 323 of the stator magnetic bearing arrangement 320 form an inlet-side passive radial magnetic bearing. The rotor magnet arrangement 210 further comprises one or more outlet-side rotor bearing magnets 213, which together with one or more outlet-side stator bearing magnets 324 of the stator magnetic bearing arrangement 320 form an outlet-side passive radial magnetic bearing. The outlet-side rotor bearing magnet or magnets 213 are arranged peripherally to the central recess 231 of the base plate 230. The trunnion 301 comprises the outlet-side stator bearing magnet or magnets 324. Due to the arrangement of the two radial magnetic bearings in the hub structure 220 and in the base plate 230, the radial magnetic bearings may, among other things, have a stabilizing effect against tilting of the rotor with respect to the axis of rotation.

The described arrangement and combination of the magnets of the rotor magnet arrangement 210 and the stator magnetic bearing arrangement 320 are exemplary. Other arrangements and/or combinations and/or the addition and/or omission of magnets are possible. For example, the motor may be arranged on the inlet side instead of the outlet side, as is the case here. The control coil may be arranged on the outlet side. The two radial magnetic bearings may be arranged in a common plane, for example on the inside and outside of the base plate, instead of being axially spaced, as is the case here, or such a bearing arrangement may be provided additionally.

The hub structure 220 protrudes from the base plate 230 in the direction of the first end 201. A protruding length l of the hub structure 220 as protruding from the base plate 230 along the axis of rotation 400 is approximately half of a total length L of the rotor 200 along the axis of rotation 400. In further examples, the protruding length may be at least a quarter, at least a third, for example between 35 percent and 40 percent or between 40 percent and 45 percent, or at least half of the total length. An outer width b of the hub structure 220 perpendicular to the axis of rotation 400 is approximately half of a total width B of the rotor 200 perpendicular to the axis of rotation 400. In other examples, the outer width may be at least one third or at least half of the total width.

The hub structure 220 is essentially rotationally symmetrical with respect to the axis of rotation 400. The hub structure 220 is substantially cylindrical in shape, with an outer cross-section of the hub structure 220 tapering towards the second end 202 and an inner cross-section of the hub structure 220 widening towards the second end 202. In further examples, the hub structure may, for example, be essentially conical in shape at least in sections or have a cross-section that varies along the axis of rotation in some other way. The hub structure 220 has one or more cavities 221 for accommodating the inlet-side rotor bearing magnet or magnets 211.

The base plate 230 is essentially disk-shaped in the form of a flattened cylinder. The base plate 230 forms a region extending fully around the axis of rotation 400 and around the central recess 231, said region having the shape of a cylindrical ring. The base plate 230 comprises one or more cavities 232 for receiving the outlet-side rotor bearing magnet or magnets 213 and the outlet-side rotor motor magnet or magnets 212.

The blading 240 comprises a plurality of blades 243, wherein the fluid channels 241 are formed between facing respective first and second side surfaces 244, 245 of adjacent blades 243. The blades 243 extend radially in a spiral-shape from a central region of the base plate 230 towards an outer region of the base plate 230. The blading 240 is thus configured to impart a radial flow component (in the direction of the outlet opening 242) to the conveyed fluid during operation. The blades 243 or their side surfaces 244, 245 are substantially perpendicular to the base plate 230 and parallel to the axis of rotation 400, but may alternatively be angled and/or curved with respect to the axis of rotation 400.

Each of the fluid passages 241 has a passage opening 246 connecting the fluid passage 241 to the cavity 310, the passage opening 246 extending between the outlet opening 242 and the hub structure 220 along a side of the fluid passage 241 facing the first end 201. The rotor 200 is thus semi-open. The passage opening 246 of each of the fluid passages 241 extends along an entire extension of the fluid passage 241 from the outlet opening 242 to the hub structure 220. Alternatively, it may be provided that the passage opening 246 of at least one, preferably each, of the fluid passages 241 extends along only a portion of the extension of the fluid passage 241 from the outlet opening 242 towards the hub structure 220. It may also be provided that only one or more, but not all, fluid channels 241 each have a passage opening 246 of the type described.

The hub structure 220 comprises an inlet channel 222 extending along the axis of rotation 400 and open at the first end 201, which is fluidly connected to the fluid channels 241 within the rotor 200. The hub structure 220 thus acts as a fluid inlet of the rotor 200 through which at least a portion, in particular a predominant portion, of the fluid entering through the inlet 311 of the cavity 310 is directed to the blading 240. The fluid passages 241 have inlet openings 247 opening into the inlet channel 222, through which the fluid may enter the fluid passages 241 from the inlet channel 222 during operation. Alternatively or additionally, it may be provided that the fluid enters the fluid channels through a gap between the hub structure and a wall of the cavity. A transition 229 from the blading 240 to the hub structure 220 is rounded. Alternatively, the transition may be designed without rounding.

A first component 250 of the rotor 200 comprising the blading 240 is formed integrally, in particular by milling from a continuous piece of material, preferably metal. The first component 250 includes a portion of the base plate 230 and a portion of the hub structure 220. The rotor 200 comprises an integrally formed second component 251 comprising a portion of the hub structure 220 disposed at the first end 201, and an integrally formed third component 252 comprising a portion of the base plate 230 disposed at the second end 202. The cavity or cavities 221 of the hub structure are formed between the first component 250 and the second component 251, and the cavity or cavities 232 of the base plate are formed between the first component 250 and the third component 252. Alternative subdivisions of the rotor 200 are possible, for example the first and/or second and/or third component may be further subdivided into integrally formed components or segments.

The fluid pump 100 may comprise a control unit for controlling (open-loop and/or closed-loop control) a rotation and/or position of the rotor 200 within the cavity 310 and/or may be connectable or connected to such a control unit. The pump 100 may include one or more sensors for providing measurement signals corresponding to parameters of the rotation and/or position of the rotor 200 and/or corresponding to other parameters, such as a pressure and/or a pressure difference and/or forces and/or torques acting on the rotor 200. The control unit may be configured to control (open-loop or closed-loop) the rotation and/or position of the rotor 200 based on the measurement signals. For example, the control unit may be configured to detect a measurement signal that may be registered at the motor coils 322, the measurement signal having a component that is dependent on the position and/or rotation of the rotor; the component may correspond in particular to a back electromotive force (Back-EMF) induced due to the rotation of the rotor 200. Furthermore, the control unit may, for example, be configured to control the rotor 200 to a target bearing position (in particular a positionally variable position) at which external forces acting on the rotor 200 along a bearing direction of action, for example along the axis of rotation, add up to a predetermined force, in particular a zero force, and/or at which a power applied to actuate the control and/or motor coils 321, 322 is minimal.

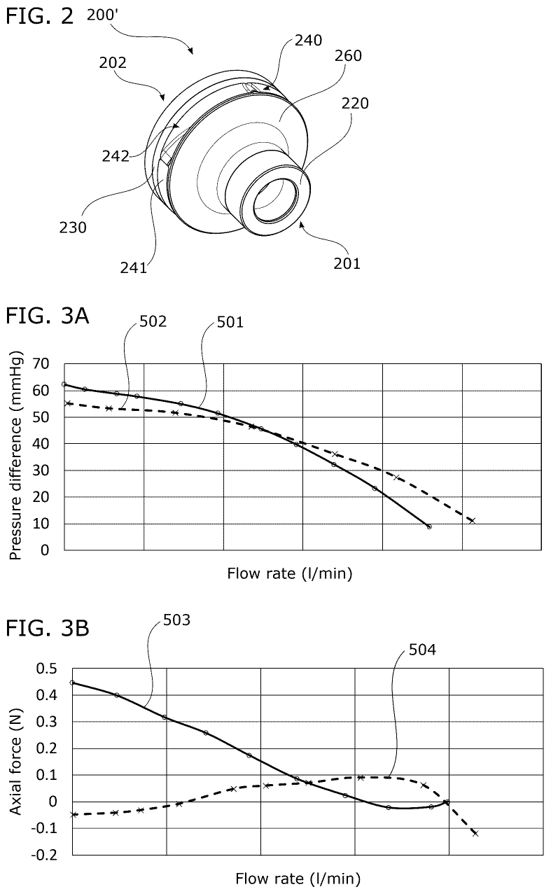

FIG. 2, which does not relate to the subject matter of the application, shows a closed rotor 200′. The closed rotor 200′ is substantially similar to the rotor shown in FIGS. 1A-FIG. 1C; in the rotor 200′, however, the fluid channels 241 are delimited towards the first end 201 by a cover plate 260 opposite the base plate 230. The fluid channels 241 thus form cavities arranged inside the rotor 200′. The complex geometry formed in this way is difficult to obtain through one-piece (integral) production, for example by milling. In addition, access to internal surfaces is difficult, which may make subsequent surface treatment more difficult. In contrast, the design of the semi-open rotor 200 enables simplified manufacture as described above (in particular due to the passage openings 246, i.e. by omitting the cover plate 260).

The characteristic curves shown in FIG. 3A and FIG. 3B correspond to measured data obtained with rotors of substantially the types shown in FIGS. 1A-1C (semi-open) and FIG. 2 (closed). The rotors were each operated in a fluid pump essentially of the type shown in FIG. 1A. Water was used as the fluid.

FIG. 3A shows a dp-Q characteristic curve 501 of a semi-open rotor of the type shown in FIG. 1A to FIG. 1C (solid graph) and a dp-Q characteristic curve of a closed rotor of the type shown in FIG. 2 (dashed graph). The dp-Q characteristic curve 501 of the semi-open rotor, has, over substantial regions, a higher slope than the dp-Q characteristic 502 of the closed rotor.

FIG. 3B shows an axial force characteristic curve 503 of a semi-open rotor of the type shown in FIG. 1A to FIG. 1C (solid graph) and an axial force characteristic curve 504 of a closed rotor of the type shown in FIG. 2 (dashed graph). The axial force characteristic curve 503 of the semi-open rotor has, over substantial regions, a steeper slope than the axial force characteristic curve 504 of the closed rotor. In addition, the axial force characteristic curve 503 of the semi-open rotor is monotonic over large regions, in particular strictly monotonic in the range of the data points measured in these regions, and is therefore in particular invertible. In contrast, the axial force characteristic curve 504 of the closed rotor has a reversal point and almost flat (i.e. not strictly monotonic) regions and is non-invertible over large regions.

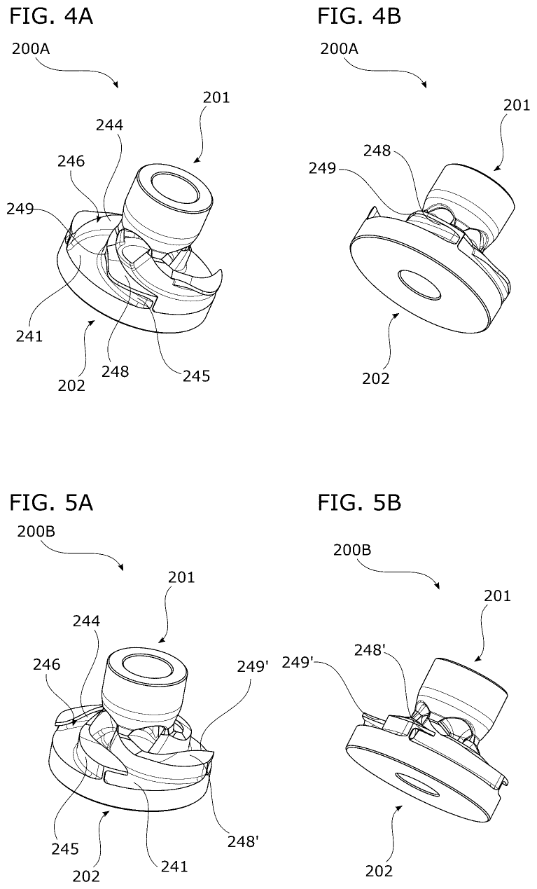

The rotor 200A shown in FIG. 4A and FIG. 4B corresponds in essential parts to the rotor 200 shown in FIG. 1A to FIG. 1C. In addition to its features, in the rotor 200A each of the fluid passages 241 is partially delimited on its side facing the first end 201 at the level of the passage opening 246 by a projection 248 (winglet) of the second side surface 245 adjacent to the respective fluid passage 241. The projection 248 projects in a direction of rotation of the rotor 200A from the convexly curved (in a plane perpendicular to the axis of rotation 400) second side surface 245. At an end facing away from the second side surface 245, the projection 248 has an edge 249 which in some areas substantially follows the curvature of the second side surface 245, but is, for example, more strongly curved in some areas. The edge 249 may also, for example, be straight in some areas and/or curved less than the second side surface 245 in some areas and/or curved opposite to the curvature of the second side surface 245 in some areas. The projection 248 projects substantially flat and perpendicularly from the second side surface 245. Alternatively, the projection 248 may be angled at least in some areas relative to the second side surface 245 or deviate from the flat shape (i.e. be curved).

The rotor 200B shown in FIG. 5A and FIG. 5B also corresponds in essential parts to the rotor 200 shown in FIG. 1A to FIG. 1C. In addition to its features, in the rotor 200B each of the fluid passages 241 is partially delimited on its side facing the first end 201 at the level of the passage opening 246 by a projection 248′ (winglet) of the first side surface 244 adjacent to the respective fluid passage 241. In contrast to the rotor 200A shown in FIG. 4A and FIG. 4B, the projection 248′ of the rotor 200B projects from the concave (in a plane perpendicular to the axis of rotation 400) curved first side surface 244 opposite to the direction of rotation of the rotor 200B. At an end facing away from the first side surface 244, the projection 248′ has an edge 249′ which essentially follows the curvature of the first side surface 244 and is curved in the opposite direction to this curvature in some areas. The edge 249′ may also, for example, be straight in some areas and/or curved less than the first side surface 244 in some areas and/or curved more than the first side surface 244 in some areas. The projection 248′ projects substantially flat and perpendicularly from the first side surface 244. Alternatively, the projection 248′ may be angled at least in some areas relative to the first side surface 244 or deviate from the flat shape (i.e. be curved).

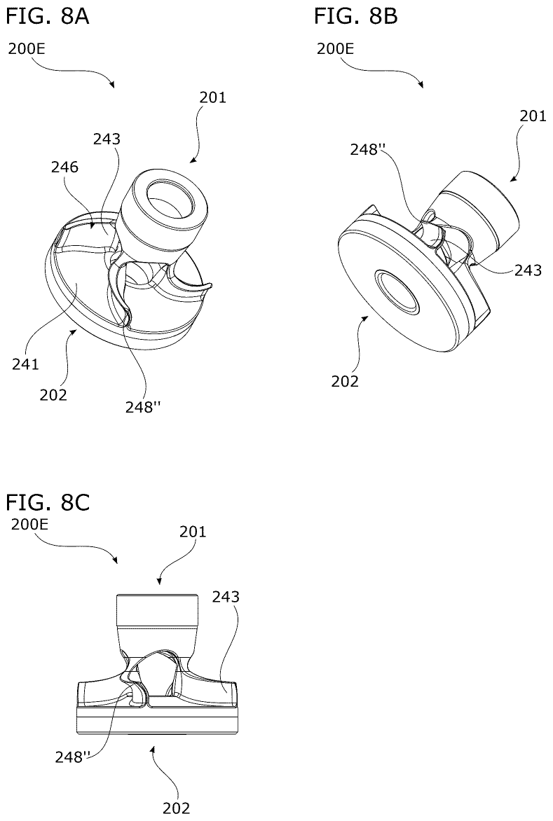

The rotor 200E shown in FIG. 8A and FIG. 8B also corresponds in essential parts to the rotor 200 shown in FIG. 1A to FIG. 1C. In addition to its features, in the rotor 200C each of the fluid passages 241 is partially delimited on its side facing the first end 201 at the level of the passage opening 246 by a projection 248″ of a surface of the respective blade 243 adjacent to the respective fluid passage 241. In contrast to the rotors 200A and 200B, in which the respective projections 248, 248′ are formed as flat cover pieces projecting perpendicularly from the respective side surface 245, 244, each of the projections 248″ of the rotor 200E is formed as a continuous extension of a shape of the respective blade 243. The blades 243 are curved in a tangential plane parallel to the rotational axis (400) in such a way that they are convexly bulging against a direction of rotation of the rotor (200, 200A, 200B, 200C, 200D, 200E). Alternatively, the blades may be convexly bulging in the direction of rotation.

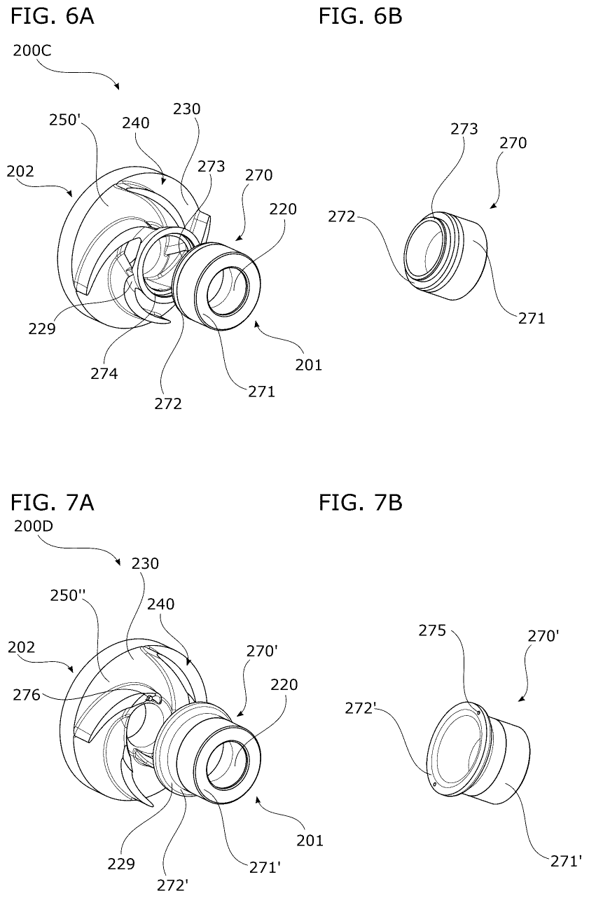

The rotor 200C shown in FIG. 6A corresponds in essential parts to the rotor 200 shown in FIG. 1A to FIG. 1C, but differs from it in the subdivision of the components of the rotor 200C. A first component 250′ of the rotor 200C comprising the blading 240 is integrally formed, in particular by milling from a continuous piece of metal material. The first component 250′ comprises at least a part of the base plate 230. The hub structure 220 is shown as a separate hub assembly 270 (shown separately from a different perspective in FIG. 6B), comprising a first hub component 271 disposed at the first end 201 and a second hub component 272 disposed between the first hub component 271 and the base plate 230. The cavity or cavities of the hub structure for receiving magnets are formed between the first hub component 271 and the second hub component 272. The rounded transition 229 is arranged at the first component 250′.

The second hub component 272 comprises a protruding alignment ring 273 facing the second end 202. The first component 250′ comprises an alignment groove 274 facing the first end 201 for receiving the alignment ring 273. Alternatively, the first component 250′ may comprise the alignment ring 273, and the second hub component 272 may comprise the alignment groove 274. The alignment ring 273 and the alignment groove 274 form a groove connection by means of which the first component 250′ and the hub assembly 270 are connectable or connected to each other.

The rotor 200D shown in FIG. 7A corresponds in essential parts to the rotor 200 shown in FIG. 1A to FIG. 1C, but differs from it in the subdivision of the components of the rotor 200D. A first component 250″ of the rotor 200C comprising the blading 240 is formed integrally, in particular by milling from a continuous piece of metal material. The first component 250″ comprises at least a part of the base plate 230. The hub structure 220 is shown as a separate hub assembly 270′ (shown separately from a different perspective in FIG. 7B), comprising a first hub component 271′ disposed at the first end 201 and a second hub component 272′ disposed between the first hub component 271′ and the base plate 230. The cavity or cavities of the hub structure for receiving magnets are formed between the first hub component 271′ and the second hub component 272′. The rounded transition 229 is arranged on the second hub component 272′.

The second hub component 272′ includes alignment holes 275 facing the second end 202. The first component 250″ comprises alignment pins 276 facing the first end 201, which may be received in respective corresponding alignment holes 275. Alternatively, the first component 250″ may comprise the alignment holes 275, and the second hub component 272′ may comprise the alignment pins 276. The alignment holes 275 and the alignment pins 276 form a pin connection by means of which the first component 250″ and the hub assembly 270′ are connectable or connected to each other.

The application also concerns the following aspects:
1. A rotor (200, 200A, 200B, 200C, 200D) for a fluid pump (100) for cardiac support, comprising
   a rotor magnet arrangement (210) for contactlessly supporting the rotor (200, 200A, 200B, 200C, 200D) within a cavity (310) of the fluid pump (100) such that the rotor (200, 200A, 200B, 200C, 200D) is rotatable about an axis of rotation (400) for conveying a fluid from an inlet (311) of the cavity (310) to an outlet (312) of the cavity (310), and a first end (201) of the rotor (200, 200A, 200B, 200C, 200D) is arranged closer to the inlet (311) than a second end (202) of the rotor (200, 200A, 200B, 200C, 200D) opposite the first end (201) along the axis of rotation (400),
   a hub structure (220) arranged at the first end (201) and centered about the axis of rotation (400), the hub structure (220) comprising one or more magnets of the rotor magnet arrangement (210),
   a base plate (230) arranged at the second end (202) or between the hub structure (220) and the second end (202) with a blading (240) for conveying the fluid, wherein intermediate spaces of the blading (240) form fluid channels (241) which open into respective outlet openings (242) and are delimited towards the second end (202) by the base plate (230),
   wherein at least one, preferably each, of the fluid channels (241) has a passage opening (246) connecting the fluid channel (241) to the cavity (310), which passage opening extends between the outlet opening (242) and the hub structure (220) at least in certain regions along a first side of the fluid channel (241) facing the first end (201).
2. A rotor (200, 200A, 200B, 200C, 200D) according to aspect 1, wherein the passage opening (246) of at least one, preferably each, of the fluid passages (241) extends along at least a portion of an extension of the fluid passage (241) from the outlet opening (242) towards the hub structure (220).
3. A rotor (200, 200A, 200B, 200C, 200D) according to aspect 2, wherein the passage opening (246) of at least one, preferably each, of the fluid passages (241) extends along the entire extension of the fluid passage (241) from the outlet opening (242) to the hub structure (220).
4. A rotor (200, 200A, 200B, 200C, 200D) according to one of the preceding aspects, wherein at least one, preferably each, of the fluid channels (241) is partially delimited on the first side at the level of the passage opening (246) by a projection (248, 248′) of a surface of the blading (240) adjacent to the respective fluid channel (241).
5. A rotor (200, 200A, 200B, 200C, 200D) according to aspect 4, wherein the projection (248, 248′) projects from the surface of the blading (240) adjacent to the respective fluid channel (241) counter to a direction of rotation of the rotor (200, 200A, 200B, 200C, 200D) and/or this surface has a concave curvature.
6. A rotor (200, 200A, 200B, 200C, 200D) according to aspect 4, wherein the projection (248, 248′) projects in a direction of rotation of the rotor (200, 200A, 200B, 200C, 200D) from the surface of the blading (240) adjacent to the respective fluid channel (241) and/or this surface has a convex curvature.
7. A rotor (200, 200A, 200B, 200C, 200D) according to any of the preceding aspects, wherein the hub structure (220) comprises an inlet channel (222) extending along the axis of rotation (400) and open at the first end (201) and fluidly connected to the fluid channels (241) within the rotor (200, 200A, 200B, 200C, 200D).
8. A rhe rotor (200, 200A, 200B, 200C, 200D) according to any one of the preceding aspects, wherein a transition (229) from the blading (240) to the hub structure (220) is rounded.
9. A rotor (200, 200A, 200B, 200C, 200D) according to one of the preceding aspects, wherein a component (250, 250′, 250″) of the rotor (200, 200A, 200B, 200C, 200D) comprising the blading (240) is formed integrally, in particular by milling from a coherent piece of material.
10. A rhe rotor (200, 200A, 200B, 200C, 200D) according to aspect 9, wherein the integrally formed component (250, 250′, 250″) comprising the blading (240) comprises at least a portion of the base plate (230) and/or at least a portion of the hub structure (220).
11. A rotor (200, 200A, 200B, 200C, 200D) according to any of the preceding aspects, wherein the rotor (200, 200A, 200B, 200C, 200D) is composed or composable of a first component comprising at least a part of the hub structure and at least one second component.
12. A rotor (200, 200A, 200B, 200C, 200D) according to any of the preceding aspects, wherein the base plate (230) comprises one or more magnets of the rotor magnet arrangement (210).
13.A rotor (200, 200A, 200B, 200C, 200D) according to one of the preceding aspects, wherein the base plate (230) comprises a central recess (231) for contactlessly receiving a part of a stator of the fluid pump (100), in particular a trunnion with a magnetic bearing component.
14. A fluid pump (100) for cardiac support, comprising
   a rotor (200, 200A, 200B, 200C, 200D) according to any of the preceding aspects, a stator (300) with a cavity (310) for receiving the rotor (200, 200A, 200B, 200C, 200D) and a stator magnetic bearing arrangement (320) which, together with the rotor magnet arrangement (210), forms a magnetic bearing, in particular an active magnetic bearing, for contactlessly supporting the rotor (200, 200A, 200B, 200C, 200D) within the cavity (310).
15. A fluid pump (100) according to aspect 14, wherein the fluid pump (100) is a centrifugal pump and/or wherein the stator (300) and the rotor (200, 200A, 200B, 200C; 200D) form an axial flux motor.

To clarify the use of and to hereby provide notice to the public, the phrases “at least one of <A>, <B>, . . . and <N>” or “at least one of <A>, <B>, . . . <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, . . . or N including any one or more element alone or the one or more element in combination with one or more of the other elements which may also include, in combination, additional elements not listed. Unless otherwise indicated or the context suggests otherwise, as used herein, “a” or “an” means “at least one” or “one or more.”

### LIST OF REFERENCE SIGNS

100 fluid pump,
200, 200A, 200B, 200C, 200D, 200E semi-open rotor,
200′ closed rotor,
201 first end,
202 second end,
210 rotor magnet arrangement,
211 inlet-side rotor bearing magnet,
212 outlet-side rotor motor magnet,
213 outlet-side rotor bearing magnet,
220 hub structure,
221 cavity,
222 inlet channel,
229 transition,
230 base plate,
231 central recess,
232 cavity,
240 blading,
241 fluid channel,
242 outlet opening,
243 blade,
244 first side surface,
245 second side surface,
246 passage opening,
247 Inlet opening,
248, 248′, 248″ projection,
249, 249′ edge,
250, 250′, 250″ first component,
251 second component,
252 third component,
260 cover plate,
270, 270′ hub assembly,
271, 271′ first hub component,
272, 272′ second hub component,
273 alignment ring,
274 alignment groove,
275 alignment hole,
276 alignment pin,
300 stator,
301 trunnion,
310 cavity,
311 inlet,
312 outlet,
313 volute,
320 stator magnetic bearing arrangement,
321 control coil,
322 motor coil,
323 inlet-side stator bearing magnet,
324 outlet-side stator bearing magnet,
400 axis of rotation
501 dp-Q characteristic curve, semi-open rotor,
502 dp-Q characteristic curve, closed rotor,
503 axial force characteristic curve, semi-open rotor,
504 axial force characteristic curve, closed rotor,
l protruding length,
L total length,
b outer width,
B total width.

1. A rotor for a fluid pump for cardiac support, comprising:
a rotor magnet arrangement for contactlessly supporting the rotor within a cavity of the fluid pump such that the rotor is rotatable about an axis of rotation for conveying a fluid from an inlet of the cavity to an outlet of the cavity and a first end of the rotor is arranged closer to the inlet than a second end of the rotor opposite the first end along the axis of rotation;
a hub structure arranged at the first end of the rotor and centered about the axis of rotation, the hub structure comprising one or more magnets of the rotor magnet arrangement;
a base plate arranged at the second end of the rotor or between the hub structure and the second end of the rotor and having a blading for conveying the fluid, wherein intermediate spaces of the blading form fluid channels which open into respective outlet openings and are delimited towards the second end of the rotor by the base plate;
wherein at least one of the fluid channels has a passage opening connecting the fluid channel to the cavity, the passage opening extending between the outlet opening and the hub structure along at least part of a first side of the fluid channel the first side facing the first end of the rotor.

2. The rotor of claim 1, wherein the passage opening of at least one of the fluid passages extends along at least part of an extension of the fluid passage from the outlet opening towards the hub structure.

3. The rotor of claim 2, wherein the passage opening of at least one of the fluid passages extends along the entire extension of the fluid passage from the outlet opening to the hub structure.

4. The rotor of claim 1, wherein at least one of the fluid passages is partially delimited on the first side at the level of the passage opening by a projection of a surface of the blading adjacent to the respective fluid passage.

5. The rotor of claim 4, wherein the projection projects from the surface of the blading adjacent to the respective fluid channel against a direction of rotation of the rotor and/or the surface has a concave curvature in a plane perpendicular to the axis of rotation.

6. The rotor of claim 4, wherein the projection projects from the surface of the blading adjacent to the respective fluid channel in a direction of rotation of the rotor and/or the surface has a convex curvature in a plane perpendicular to the axis of rotation.

7. The rotor of claim 4, wherein the projection is formed as a continuous extension of a shape of a respective blade of the blading, wherein the blade and/or the projection is at least partially curved in a tangential plane parallel to the axis of rotation.

8. The rotor of claim 1, wherein the hub structure comprises an inlet channel extending along the axis of rotation and open at the first end and fluidly connected within the rotor to the fluid channels.

9. The rotor of claim 8, wherein the base plate comprises a region extending fully around the axis of rotation the region essentially having a cylindrical shape.

10. The rotor of claim 1, wherein a transition from the blading to the hub structure is rounded.

11. The rotor of claim 1, wherein a component of the rotor comprising the blading is integrally formed by milling from a continuous piece of material.

12. The rotor of claim 11, wherein the integrally formed component comprising the blading comprises at least a part of the base plate and/or at least a part of the hub structure.

13. The rotor of claim 1, wherein the rotor is composed or composable of a first component comprising at least a part of the hub structure and at least one second component.

14. The rotor of claim 1, wherein the base plate comprises one or more magnets of the rotor magnet arrangement.

15. The rotor of claim 14, wherein one or more of the one or more magnets of the rotor magnet arrangement comprised by the base plate is adapted to cooperate with a stator magnetic bearing arrangement of the fluid pump for exerting a force along the axis of rotation, on the rotor for contactlessly supporting the rotor within the cavity.

16. The rotor of claim 1, wherein the base plate comprises a central recess for contactlessly receiving a part of a stator of the fluid pump, in particular a trunnion comprising a magnetic bearing component.

17. A fluid pump for cardiac support, comprising:
a rotor of claim 1,
a stator with a cavity for receiving the rotor and a stator magnetic bearing arrangement which, together with the rotor magnet arrangement, forms an active magnetic bearing for contactlessly supporting the rotor within the cavity.

18. The fluid pump according to claim 17, wherein the fluid pump is a centrifugal pump.

19. The fluid pump according to claim 17, wherein the stator and the rotor form an axial flux motor.
